(19) United States
(12) Patent Application Publication
(10) Pub. No.: US 2011/0152541 A1
(43) Pub. Date: Jun. 23, 2011

(54) INDUCING LAYER MATERIALS FOR WEAK EPITAXIAL FILMS OF NON-PLANAR METAL PHTHALOCYANINE

(76) Inventors: Donghang YAN, Shanghai (CN); Yanhou Geng, Shanghai (CN); Hongkun Tian, Shanghai (CN); Feng Pan, Shanghai (CN); Lizhen Huang, Shanghai (CN)

(21) Appl. No.: 12/854,949

(22) Filed: Aug. 12, 2010

(30) Foreign Application Priority Data
Dec. 22, 2009 (CN) 200910200459.5

(51) Int. Cl. C07D 333/76; C07D 495/04; C07D 409/14; C07D 333/10; C07D 409/04; C07C 25/18; C07C 25/22; C07C 13/54

(52) U.S. Cl. 549/43; 549/50; 549/59; 549/80; 570/129; 585/26

(57) ABSTRACT

The present invention relates to inducing layer materials for the preparation of weak epitaxial films of non-planar metal phthalocyanine. The characteristics of the inducing layer materials lies in that the said inducing layer materials replace benzene rings of sexiphenyl by conjugated aromatic group and to replace the hydrogen atoms of benzene rings at the two ends of sexiphenyl by fluorine atoms, thus the regulation of molecular interaction is finally realized by changing the size or the linearity degree of the conjugated aromatic groups, as well as by changing the polarity of the benzene ring at the two ends, consequently, the cell parameters of (001) crystal face of the new materials are different from that of sexiphenyl and to achieve a effect of inducing the weak epitaxy growth of non-planar metal-phthalocyanine.

### TECHNICAL FIELD

The present invention relates to inducing layer materials for the preparation of weak epitaxial films of non-planar metal phthalocyanine.

### BACKGROUND TECHNOLOGY

In recent years, the development of organic semiconductor materials with high carrier mobility is very active, and the materials show some broad application prospects in information display, integrated circuit, photovoltaic cells and sensors, etc. Wang Haibo, etc. (Adv. Mater., 2007, 19, 2168-2171) first reported a method to prepare polycrystalline thin films of discoid organic semiconductor on the amorphous substrate—Weak Epitaxy Growth, and the field—effect carrier mobility of the polycrystalline thin films thereof reaches the level of corresponding single crystal. The principle of Weak Epitaxy Growth is to prepare a layer of organic ultra-thin film with highly ordered arrangement as inducing layer on amorphous substrate firstly, and the discoid organic semiconductor molecules nucleated orientationally on the inducing layer, and form a high quality polycrystalline thin film with large crystal domain size, highly oriented domain and good coalescene of polycrystal domain. Wang Tong, etc. (J. Phy. Chem. B, 2008, 112, 6786-6792.) reported the Weak Epitaxy Growth behavior of planar metal phthalocyanine on the para-hexaphenyl inducing layer. The planar metal phthalocyanine shows two sets of orientation with incommensurate epitaxy and one set of orientation with commensurate epitaxy on single molecular inducing layer, and shows one set of orientation with commensurate epitaxy on bimolecular inducing layer. These growth habits have a close relationship with the lattice mismatch between the epitaxial crystal and the inducing layer. Wang Haibo, etc. (Appl. Phys. Lett., 2007, 90, 253510.) reported the Weak Epitaxy Growth of vanadyl phthalocyanine which belonging to non-planar metal phthalocyanine on para-hexaphenyl inducing layer, wherein incommensurate epitaxy between them was found. There are significant differences in parameter and type of the unit cell between non-planar metal phthalocyanine and planar metal phthalocyanine, and their Weak Epitaxy Growth habits on para-hexaphenyl inducing layer are significantly changed, so the lattice matching between them is difficult. In order to obtain high-quality polycrystalline thin films of organic semiconductors of non-planar metal phthalocyanine, it is needed to develop a new type of inducing layer materials whose lattice can be matched with that of the non-planar metal phthalocyanine.

### CONTENT OF THE INVENTION

In order to overcome the deficiencies of the prior art, the purpose of the present invention is to provide a series of new type of inducing layer materials which are suitable for the Weak Epitaxy Growth of non-planar metal-phthalocyanine.

The principle of the present invention is to replace benzene rings of para-hexaphenyl by conjugated aromatic groups (polycyclic aromatic compounds or oligomers with 2 to 4 aromatic rings containing benzene ring and thiophene ring) and to replace the hydrogen atoms of benzene rings at the two ends of para-hexaphenyl by fluorine atoms, thus the regulation of molecular interaction is finally realized by changing the size or the linearity degree of the conjugated aromatic groups, as well as by changing the polarity of the benzene ring at the two ends, consequently, the cell parameters of (001) crystal face of the new materials are different from that of para-hexaphenyl and can match the cell parameters of non-planar metal phthalocyanine. The molecular structure of the new inducing layer materials is shown as the following general formula:

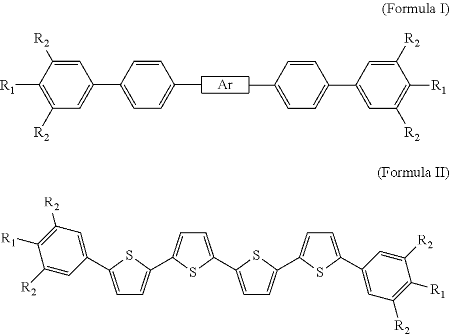

Ar in general formula I is a conjugated aromatic group, including the following structures:

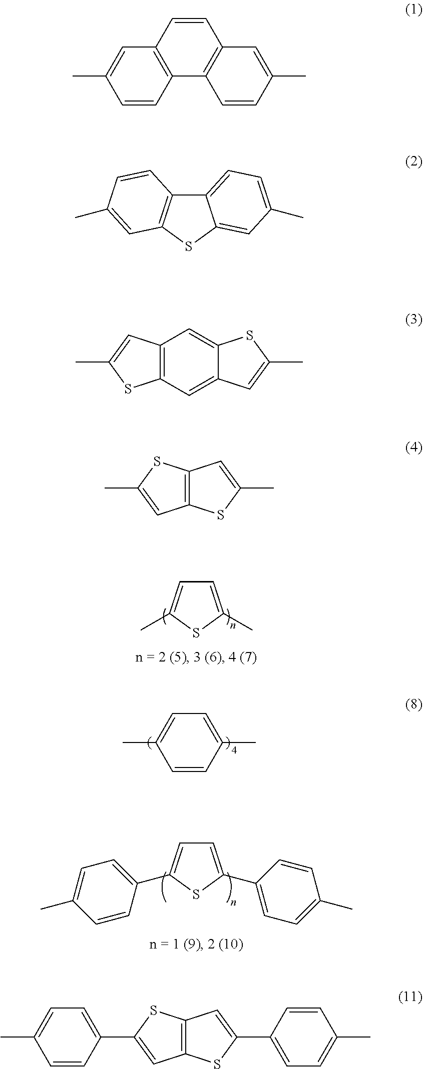

R₁ and R₂ in general formula I and II are hydrogen atoms (H) or fluorine atoms (F). According to the differences of the chemical structures of R₁ and R₂, the molecular structures of the new inducing layer materials conformed to general formula I can be divided into the following three types:

(1) R₁═R₂═H, the specific chemical structures, names and initials of the compounds are listed in Table 1.

TABLE 1

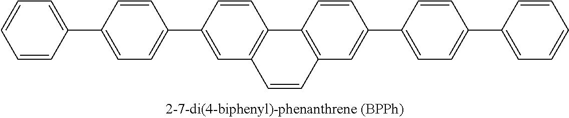

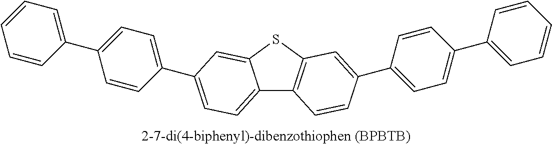

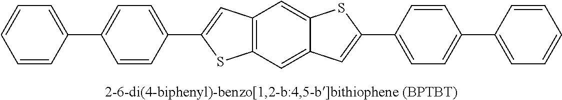

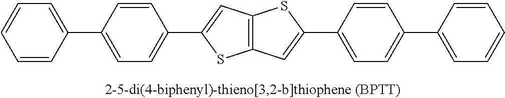

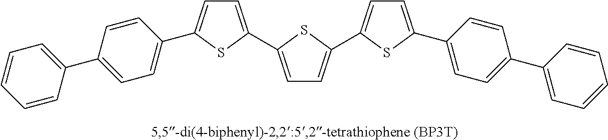

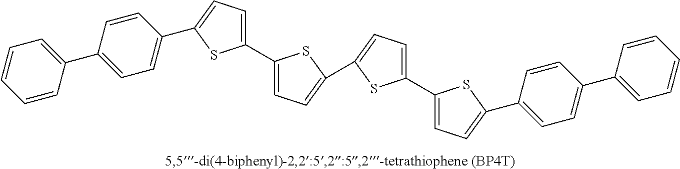

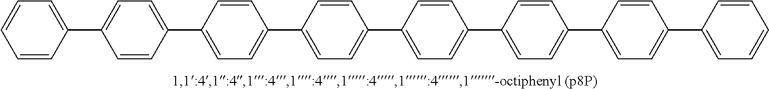

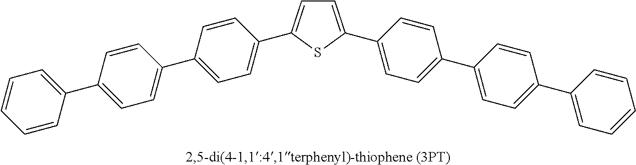

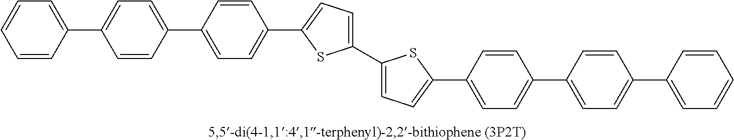

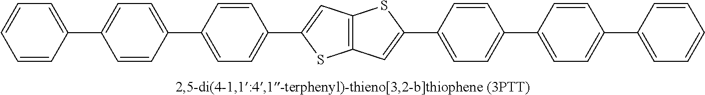

(2) R₁═F, R₂═H, specific chemical structures, names and initials of the compounds are listed in Table 2.

TABLE 2

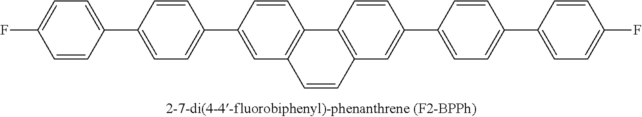

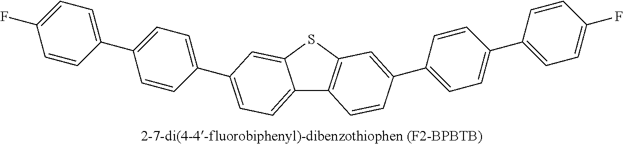

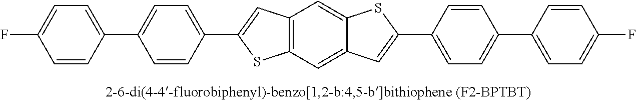

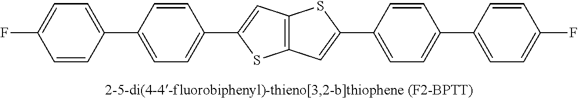

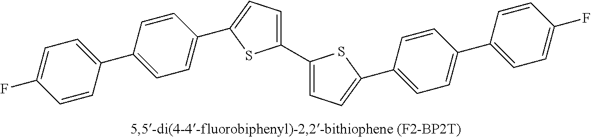

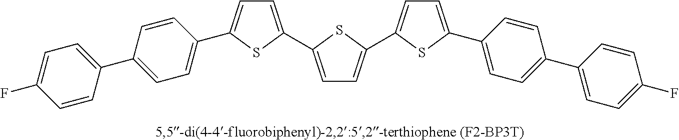

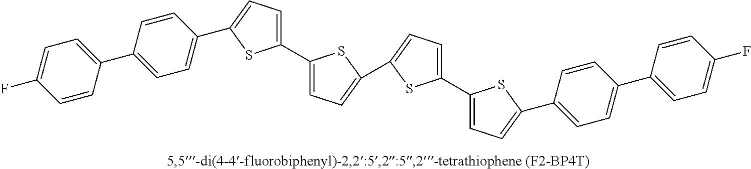

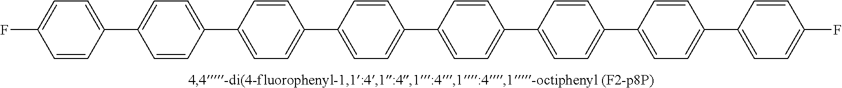

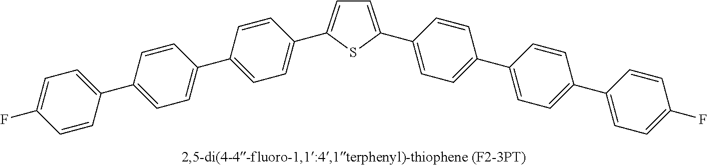

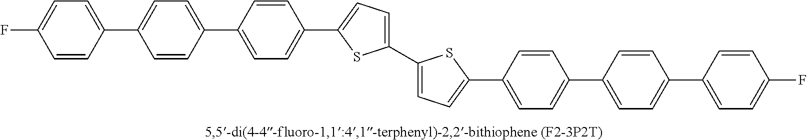

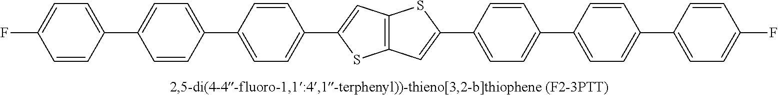

(3) R₁═H, R₂═F, specific chemical structures, names and initials of the compounds are listed in Table 3.

TABLE 3

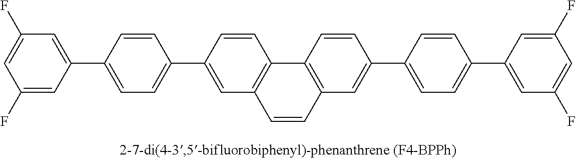

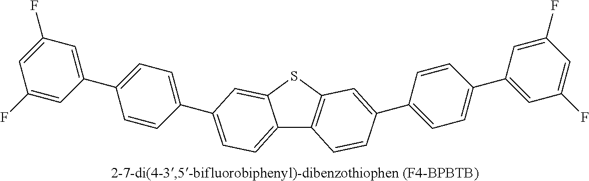

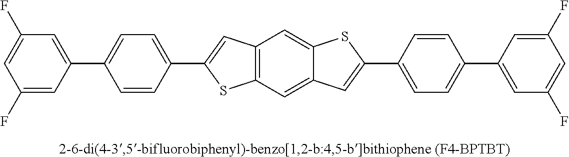

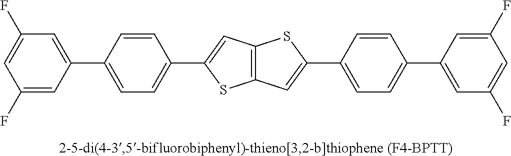

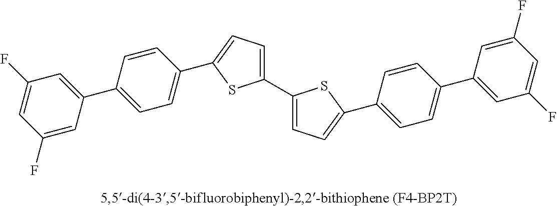

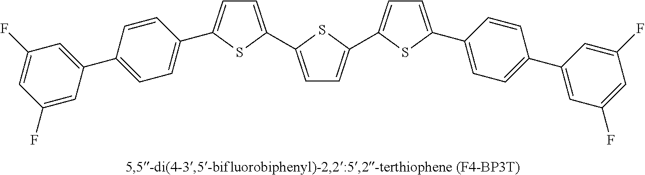

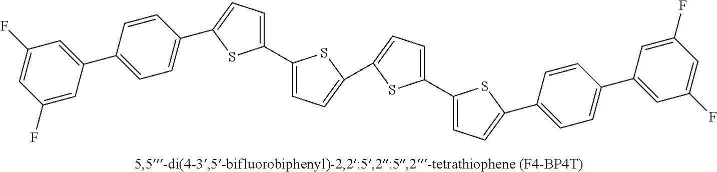

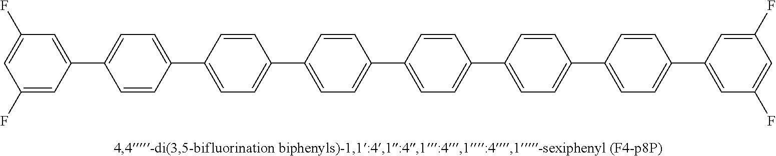

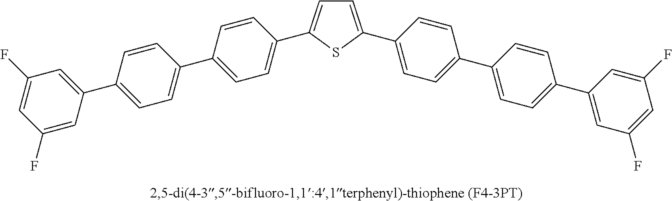

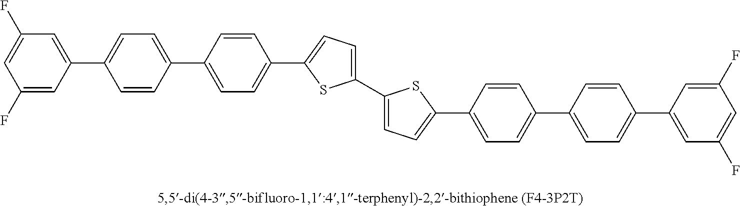

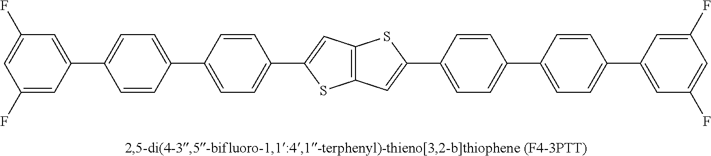

BPTT, BP3T, BP4T and p8P listed in Table 1 are prepared in accordance with the technical solutions in the prior art, as follows: BPTT (Chem. Mater., 2005, 17, 3861-3870.), BP3T (J. Heterocycl. Chem., 2000, 37, 281-286.), BP4T (J. Heterocycl. Chem., 2003, 40, 845-850.) and p8P (Org. Biomol. Chem., 2004, 2, 452-454.). BPPh, BPBTB and BPTBT can be produced by the Suzuki coupling reaction, and the reaction intermediates involved are industrial products or prepared in accordance with the technical solutions in the prior art, as follows: 4-biphenyl boric acid is industrial product, 2,7-dibromophenanthrene (Chem. Commun., 2006, 3498-3500.), 2,7-dibromo dibenzothiophen (J. Mater. Chem., 1999, 9, 2095-2101.), 2,6-diiodo-benzo[1,2-b: 4,5-b′]bithiophene (J. Org. Chem., 1994, 59, 3077-3081.). 3PT, 3P2T and 3PTT can be produced by Stille coupling reaction, and the reaction intermediates involved are industrial products or prepared in accordance with the technical solutions in the prior art, as follows: 4-bromo-1,1′:4′,1″-terphenyl (J. Phys. Chem. B, 2001, 105, 8845-8860.), 2,5-di(tributylstannyl)thiophene and 5,5′-di-(tributylstannyl)-2,2′-bithiophene (J. Org. Chem., 1995, 60, 6813-6819.), 2,5-di(tributylstannyl)-thieno[3,2-b]thiophene (J. Chem. Soc., Perkin. Trans. 1, 1997, 15, 3465-3470.).

The preparation of the compounds listed in Table 2 involves a key intermediate 4-bromo-4′-fluorobiphenyl, which prepared in accordance with technical solutions in the prior art (J. Chem. Soc., 1937, 1359). As shown in the following drawing, through the chemical reaction between 4-bromo-4′-fluorination biphenyl and magnesium metal, the corresponding grignard reagent can be prepared, then the grignard reagent reacts with trimethyl borate, the product is finally acidified with dilute hydrochloric acid and 4′-fluorination biphenyl-4-boric acid is obtained.

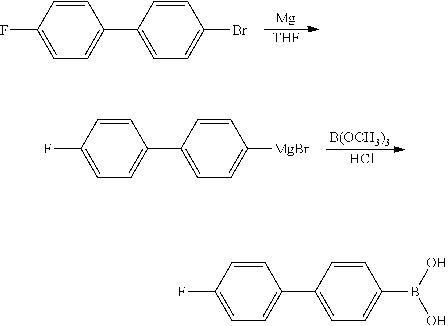

The preparation method of F2-BPPh, F2-BPBTB, F2-BPTBT is the same as that of BPPh, BPBTB, BPTBT respectively, it is only needed to replace 4-biphenyl boric acid by 4′-fluorobiphenyl-4-boric acid. F2-BPTT, F2-BP2T, F2-BP3T can be produced through Stille coupling reaction between 4-bromo-4′-fluoriobiphenyl and 2,5-di(tributylstannyl)-thieno[3,2-b]thiophene, 5,5-di(tributylstannyl)-2,2′-bithiophene and 5,5″-di(tributylstannyl)-2,2′:5′,2″-terthiophene respectively (J. Org. Chem., 1995, 60, 6813-6819.). The preparation process of F2-BP4T is shown as the following drawing: 2-(4-4′-fluorobiphenyl)thiophene can be produced by Kumada coupling reaction between 4-bromo-4′-fluorobiphenyl and the grignard reagent of thiophene, then the 2-(4-4′-fluorobiphenyl)thiophene reacts with NBS to produce 5-bromo-2-(4-4′-fluorobiphenyl). Finally F2-BP4T can be produced by Stille coupling reaction between 5-bromo-2-(4-4′-fluorobiphenyl)thiophene and 5,5′-di (tributylstannyl)-2,2′-bithiophene.

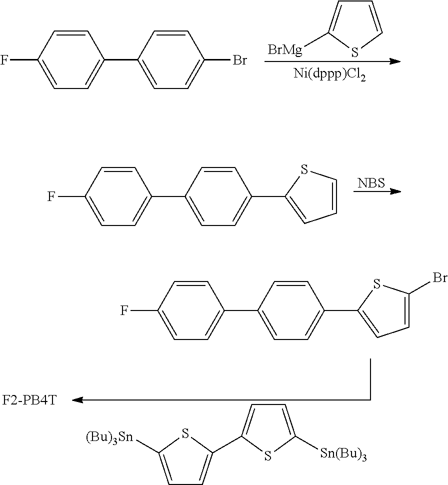

The preparation process of F2-p8P is shown as the following drawing: 4-bromo-4′″-fluoro-1,1′:4′,1″:4″,1′″-p-quaterphenyl can be produced by Suzuki coupling reaction between 4′-fluorobiphenyl-4-boric acid and 4,4′-dibromo biphenyl (industrial product), the product is purified by sublimating and used in the preparation of F2-p8P through Yamamoto coupling reaction.

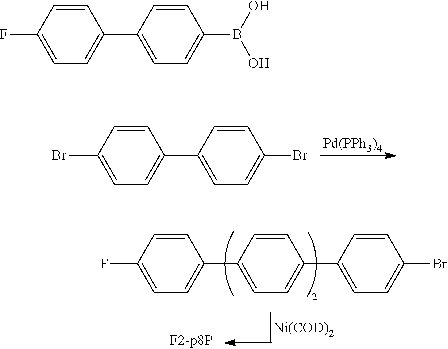

The preparation methods of F2-3PT, F2-3P2T and F2-3PTT are as follows: 4-bromo-4″-fluoro-1,1′:4′,1″-terphenyl can be produced by Suzuki coupling reaction between 4′-fluorobiphenyl-4-boric acid and 1-bromo-4-iodobenzene (industrial product) (as the following drawing), the product reacts through Stille coupling reaction with 2,5-di(tributylstannyl)thiophene and 5,5′-di(tributylstannyl)-2,2′-bithiophene and 2,5-di(tributylstannyl)-thieno[3,2-b]thiophene respectively, thus F2-3PT, F2-3P2T and F2-3PTT are correspondingly obtained 5′.

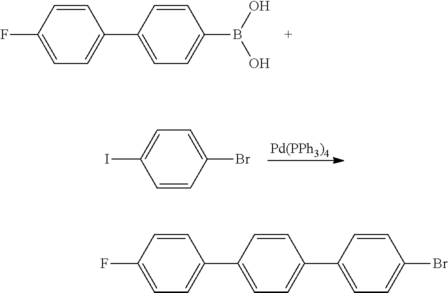

The preparations of the compounds listed in Table 3 involve a key intermediate 4-bromo-3′,5′-difluorobiphenyl, which is prepared in accordance with the technical solution in the prior art (J. Organomet. Chem., 2000, 598, 127-135.) as shown in the following drawing: the corresponding grignard reagent can be prepared through the chemical reaction between 3,5-difluoro bromobenzene (industrial product) and magnesium metal, then the grignard reagent reacts with trimethyl borate, the product is finally acidified with dilute hydrochloric acid to prepare 3,5-difluorobenzene boric acid, and 4-bromo-3′,5′-difluorobiphenyl can be produced by Suzuki coupling reaction between 3,5-difluorobenzene boric acid and 1-bromo-4-iodobenzene (industrial product).

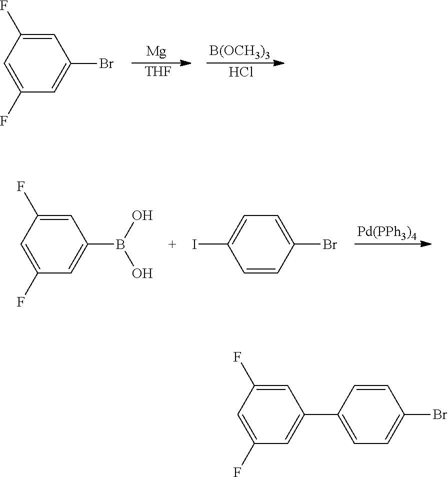

According to the preparation method of 4′-fluorobiphenyl-4-boric acid provided above, 3′,5′-difluorobiphenyl-4-boric acid can be produced by replacing 4-bromo-4′-fluoro biphenyl with 4-bromo-3′,5′-difluoro biphenyl.

In view of the structural differences between the compounds listed in Table 2 and Table 3 being 4′-fluorophenyl and 3′,5′-difluorophenyl, on the premise that 4-bromo-3′,5′-difluorobiphenyl and 3′,5′-difluorobiphenyl-4-boric acid have been obtained, all the compounds listed in Table 3 are easy to be prepared by using the synthetic method of the compounds listed in Table 2.

The specific chemical structures, names and initials of the compounds of the new inducing layer materials, conformed to general formula II, are listed in Table 4.

TABLE 4

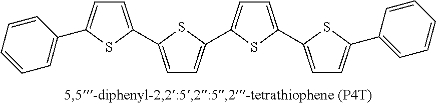

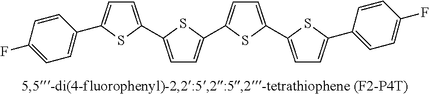

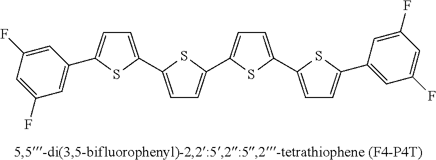

The preparation process of P4T, F2-P4T and F4-P4T is shown as the following drawing: 2-phenylthiophene, 2-(4-fluorophenyl)thiophene or 2-(3,5-difluorophenyl)thiophene can be produced by Kumada coupling reactions between the grignard reagent of thiophene and bromobenzene, 4-bromofluorobenzene or 3,5-difluorobromobenzene respectively, then 2-phenylthiophene, 2-(4-fluorophenyl)thiophene or 2-(3,5-difluorophenyl)thiophene reacts with NBS respectively, the corresponding brominated products, namely, 2-bromo-5-phenylthiophene, 2-bromo-5-(4-fluorophenyl)thiophene or 2-bromo-5-(3,5-difluorophenyl)thiophene are obtained, P4T, F2-P4T or F4-P4T can be finally produced through Stille coupling reactions between 5,5′-di(tributylstannyl)-2,2′-bithiophene and the above-mentioned brominated products respectively.

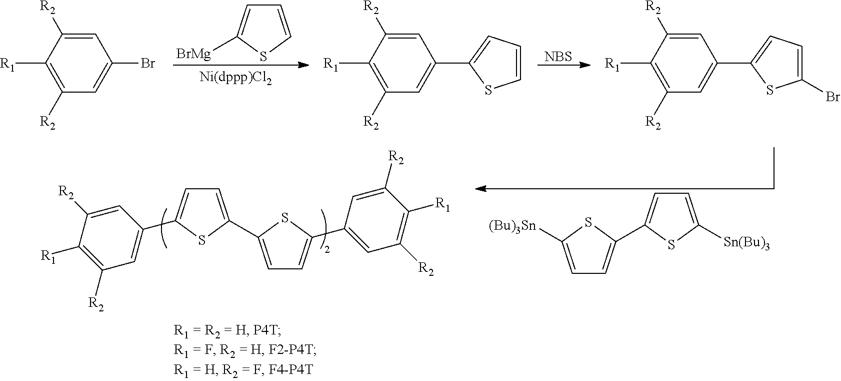

The final pure products of all the above-mentioned inducing layer materials are produced by vacuum sublimation.

### DESCRIPTION OF FIGURES

FIG. 1a is an atomic force microscope image of BPTT inducing layer film, FIG. 1b is an atomic force microscope image of the thin film of VOPc grew on said BPTT inducing layer film, FIG. 1c is a selected area electron diffraction (SAED) pattern of the thin film of VOPc grew on said BPTT inducing layer film. Wherein the crystal face diffraction point of VOPc (−2−12) is coincident with that of BPTT (200), and there is a commensurate epitaxy relation between VOPc and BPTT in the first orientation and an incommensurate epitaxy relation in the second orientation.

FIG. 2a is an atomic force microscope image of the thin film of PbPc grew on said BPTT inducing layer film, FIG. 2b is a SAED pattern of the thin film of PbPc grew on said BPTT inducing layer film. Wherein PbPc shows a commensurate epitaxy relation with BPTT in the first orientation, and shows an incommensurate epitaxy relation in the second or the third orientation.

FIG. 3a is an atomic force microscope image of the thin film of TiOPc grew on said BPTT inducing layer film, FIG. 3b is a SAED pattern of the thin film of TiOPc grew on said BPTT inducing layer film. Wherein TiOPc shows a commensurate epitaxy relation with BPTT in the first orientation, and shows an incommensurate epitaxy relation in the second or the third orientation.

FIG. 4a is an atomic force microscope image of the thin film of SnPc grew on said BPTT inducing layer film, FIG. 4b is a SAED pattern of the thin film of SnPc grew on said BPTT inducing layer film, in which SnPc shows a commensurate epitaxy relation with BPTT.

FIG. 5a is an atomic force microscope image of F2-BP4T inducing layer film, FIG. 5b is an atomic force microscope image of the thin film of PbPc grew on said F2-BP4T inducing layer film, FIG. 5c is a SAED pattern of the thin film of PbPc grew on said F2-BP4T inducing layer film. Wherein PbPc shows a commensurate epitaxy relation with F2-BP4T in the first orientation, and shows an incommensurate epitaxy relation in the second or the third orientation.

FIG. 6a is an atomic force microscope image of the thin film of VOPc grew on F2-BP4T inducing layer film, FIG. 6b is a SAED pattern of the thin film of VOPc grew on F2-BP4T inducing layer film. VOPc shows incommensurate epitaxy relations with F2-BP4T in the first, the second and the third orientations.

FIG. 7a is an atomic force microscope image of the thin film of TiOPc grew on said F2-BP4T inducing layer film, FIG. 7b is a SAED pattern of the thin film of TiOPc grew on said F2-BP4T inducing layer film. TiOPc shows an incommensurate epitaxy relation with F2-BP4T.

### SPECIFIC WAY OF IMPLEMENTATION

The present invention is further described by combining with the example below.

### Example

The KANGNING 7059 glass substrate used in the experiment is commercial product, which is used after cleaned. The non-planar metal phthalocyanine used in the experiment is commercial product, which is used after purified by sublimation. The inducing layer material used in the experiment is used after purified by sublimation

First of all, silicon nitride whose thickness is about 300 nanometer grows by chemical vapor deposition method on KANGNING 7059 glass substrate. Then an inducing layer thin film whose thickness is 1 to 3 molecular layers deposited on the surface of the silicon nitride by molecular vapor deposition method, vacuum being 10⁻⁴ pa, substrate temperature being approximately 230° C. Finally, a layer of non-planar metal phthalocyanine is deposited continuously on the inducing layer, vacuum and substrate temperature are the same as that of the inducing layer preparation.

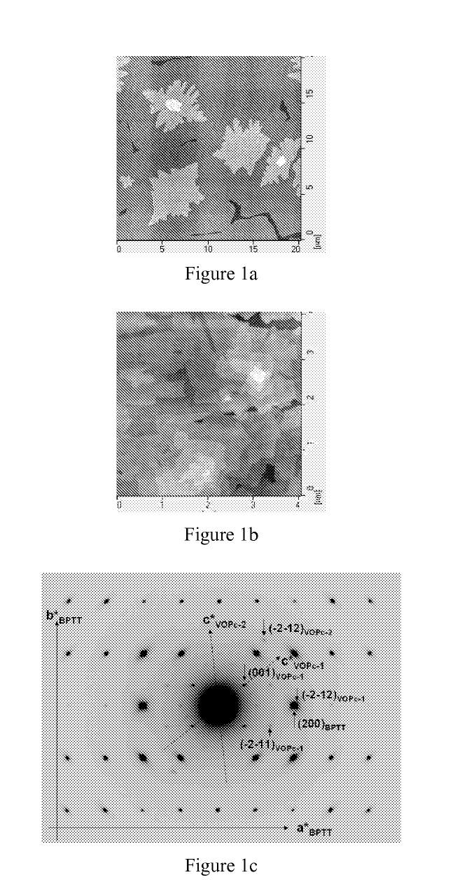

FIG. 1a is an atomic force microscope image of 2,5-di(4-biphenyl)-thieno[3,2-b]thiophene (BPTT) film with a thickness of one molecular layer, the film shows a smooth surface and is excellent for epitaxial growth of organic semiconductor layer.

FIG. 1b is an atomic force microscope image of vanadyl phthalocyanine (VOPc) thin film with a thickness of 20 nanometers formed on the surface of said film in FIG. 1a, the surface step of the VOPc molecular layer is distinct, and the VOPc film is a film with typical layer growth mode. FIG. 1c is a selected area electron diffraction (SAED) pattern of the thin film of VOPc in FIG. 1b, VOPc shows two orientations on BPTT. In the first orientation the crystal face diffraction point of VOPc (−2−12) is coincident with that of BPTT (200), which reveal a commensurate epitaxy relation between VOPc and BPTT. While in the second orientation, it is an incommensurate epitaxy relation between the VOPc and BPTT.

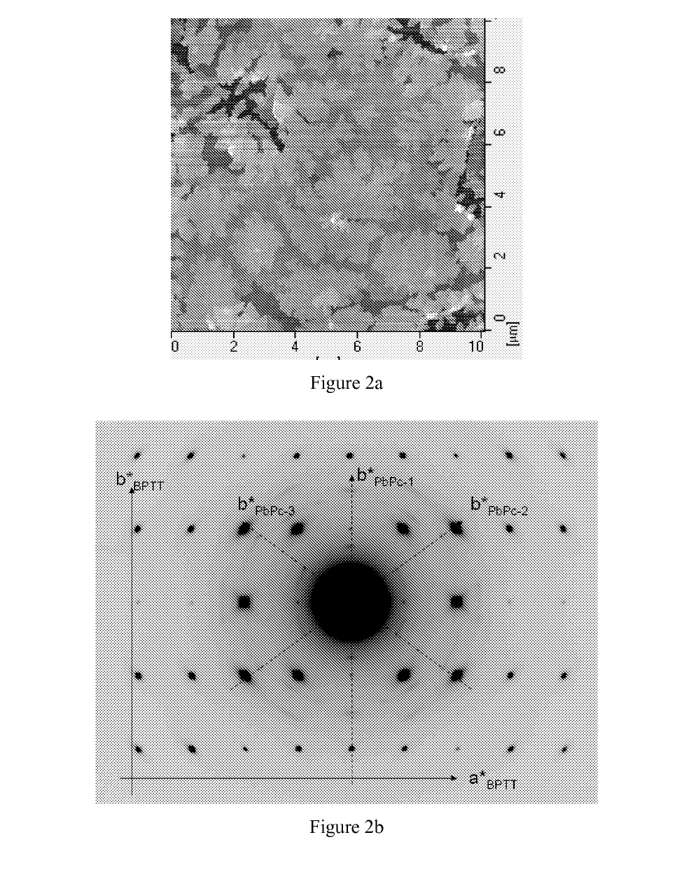

FIG. 2a is an atomic force microscope image of lead phthalocyanine (PbPc) thin film with a thickness of 20 nanometers formed on the surface of said film in FIG. 1a, the surface step of the PbPc molecular layer is distinct, and the PbPc film is a film of typical layer growth mode. FIG. 2b is a SAED pattern of the thin film of PbPc in FIG. 2a, PbPc shows three orientations on BPTT. Wherein, there is a commensurate epitaxy relation between PbPc and BPTT in the first orientation and an incommensurate epitaxy relation in the second or the third orientation.

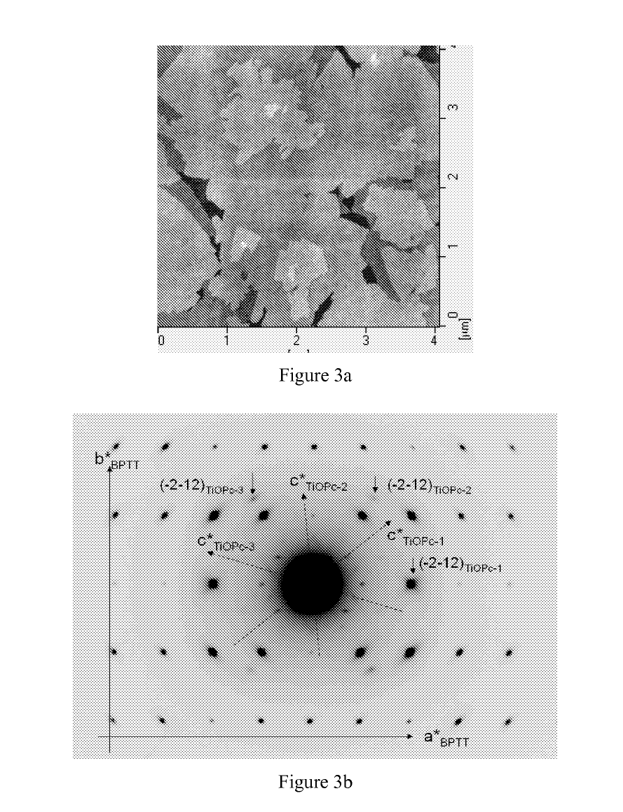

FIG. 3a is an atomic force microscope image of titanyl phthalocyanine (TiOPc) thin film with a thickness of 20 nanometers formed on the surface of said film in FIG. 1a, the surface step of the TiOPc molecular layer is distinct, and the TiOPc film is a film of typical layer growth mode. FIG. 3b is a SAED pattern of the thin film of TiOPc in FIG. 3a, TiOPc shows three orientations on BPTT. Wherein, there is a commensurate epitaxy relation between TiOPc and BPTT in the first orientation and an incommensurate epitaxy relation in the second or the third orientation.

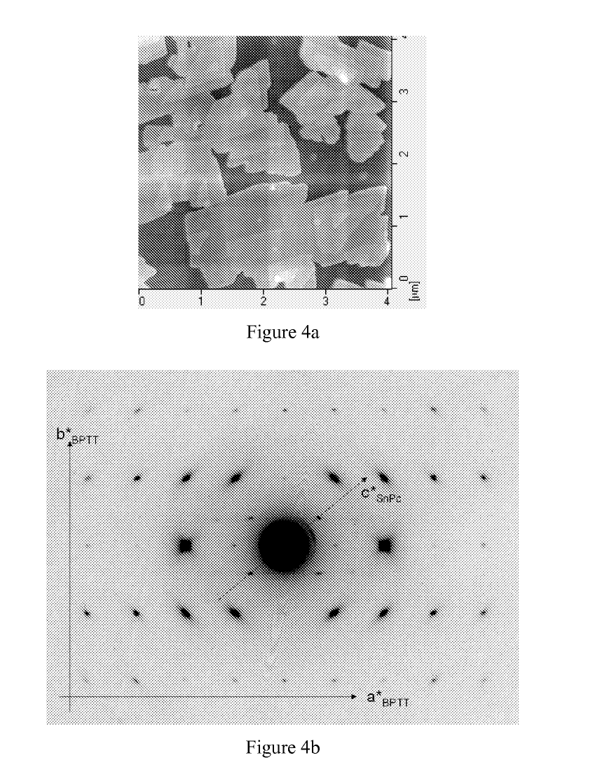

FIG. 4a is an atomic force microscope image of tin phthalocyanine (SnPc) thin film with a thickness of 20 nanometers formed on the surface of said film in FIG. 1a, the surface step of the SnPc molecular layer is distinct, and the SnPc film is a film of typical layer growth mode. FIG. 4b is a SAED pattern of the thin film of SnPc in FIG. 4a, and a commensurate epitaxy relation is shown between SnPc and BPTT.

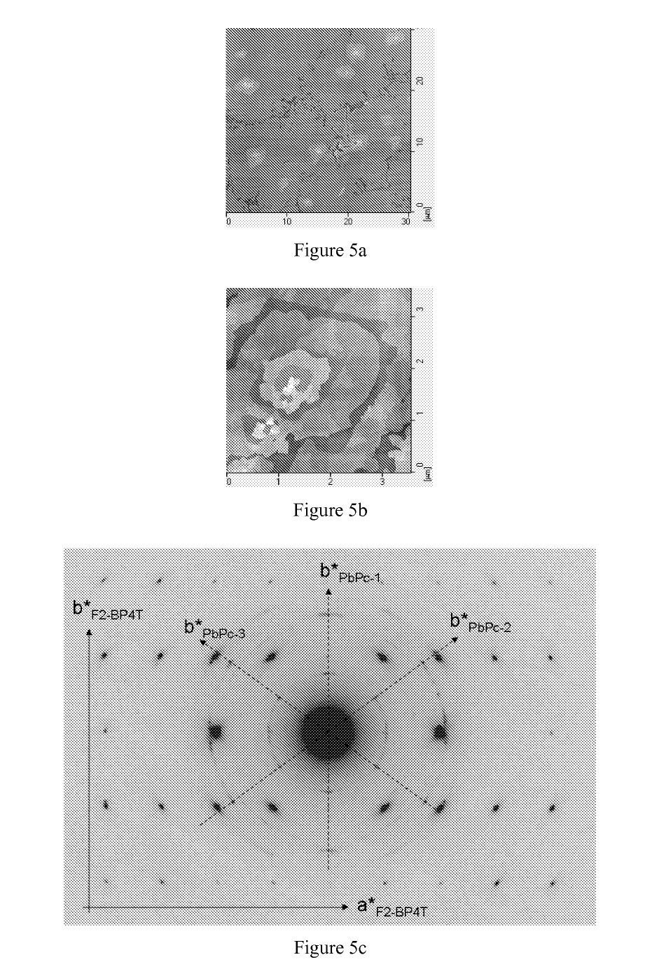

FIG. 5a is an atomic force microscope image of 5,5′″-di(4-4′-fluorobiphenyl)-2,2′:5′,2″:5″,2′″-tetrathiophene (F2-BP4T) film with a thickness of one molecular layer, the film shows a smooth surface and is excellent for the epitaxial growth of organic semiconductor layer.

FIG. 5b is an atomic force microscope image of lead phthalocyanine (PbPc) thin film with a thickness of 20 nanometers formed on the surface of said film in FIG. 5a, the surface step of the PbPc molecular layer is distinct, and the PbPc film is a film of typical layer growth mode. FIG. 5c is a SAED pattern of the thin film of PbPc in FIG. 5b, PbPc shows three orientations on F2-BP4T. Wherein, there is a commensurate epitaxy relation between PbPc and F2-BP4T in the first orientation and an incommensurate epitaxy relation in the second orientation or in the third orientation.

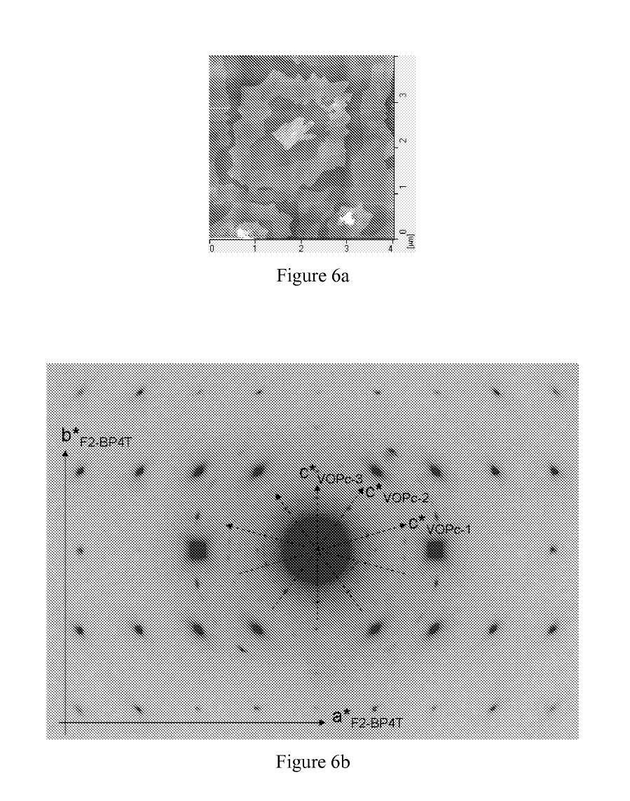

FIG. 6a is an atomic force microscope image of vanadyl phthalocyanine (VOPc) thin film with a thickness of 20 nanometers formed on the surface of said film in FIG. 5a, the surface step of the VOPc molecular layer is distinct, and the VOPc film is a film of typical layer growth mode. FIG. 6b is a SAED pattern of the thin film of VOPc in FIG. 6a, VOPc shows three orientations on F2-BP4T, which are all incommensurate epitaxy relations.

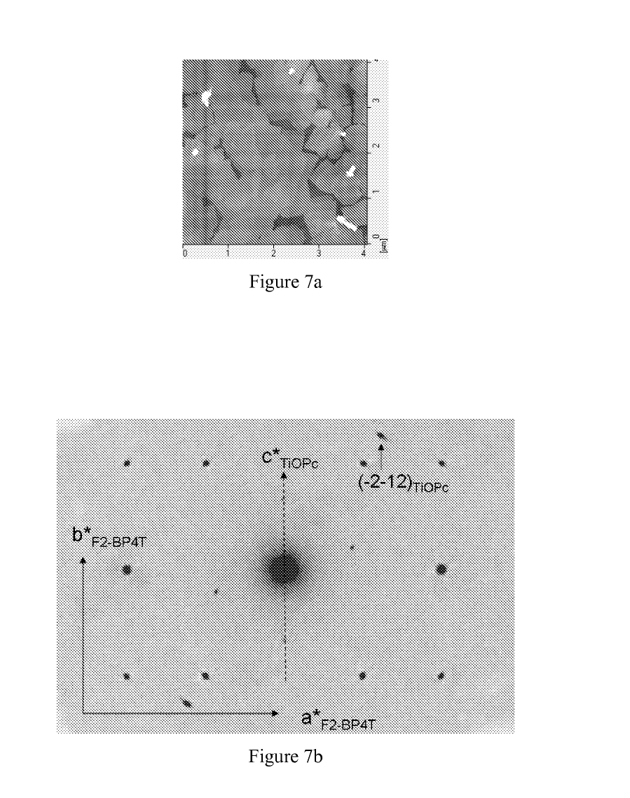

FIG. 7a is an atomic force microscope image of titanyl phthalocyanine (TiOPc) thin film with a thickness of 20 nanometers formed on the surface of said film in FIG. 5a, the surface step of the TiOPc molecular layer is distinct, and the TiOPc film is a film of typical layer growth mode. FIG. 7b is a SAED pattern of the thin film of TiOPc in FIG. 7a, there is an incommensurate epitaxy relation between TiOPc and F2-BP4T.

The epitaxial relationships between non-planar metal phthalocyanine and any one of the following inducing layer molecules are listed in Table 5:2,7-di(4-biphenyl)-phenanthrene (BPPh), 2,7-di(4-biphenyl)-dibenzothiophen (BPBTB), 2,6-di(4-biphenyl)-benzo[1,2-b:4,5-b′]bithiophene (BPTBT), 2,5-di(4-biphenyl)-thieno[3,2-b]thiophene (BPTT), 5,5″-di(4-biphenyl)-2,2′:5′,2″-terthiophene (BP3T), 5,5′″-di(4-biphenyl)-2,2′:5′,2″:5″,2′″-tetrathiophene (BP4T), 1,1′:4′,1″:4″,1′″:4′″,1″″:4″″,1′″″:4′″″,1″″″:4″″″,1′″″″-octiphenyl (p8P), 2,5-di(4-1,1′:4′,1″-terphenyl)-thiophene (3PT), 5,5′-di(4-1,1′:4′,1″-terphenyl)-2,2′-bithiophene (3P2T), 2,5-di(4-1,1′:4′,1″-terphenyl)-thieno[3,2-b]thiophene (3PTT), 2,7-di(4-4′-fluorobiphenyl)-phenanthrene (F2-BPPh), 2,7-di(4-4′-fluorobiphenyl)-dibenzothiophen (F2-BPBTB), 2,6-di(4-4′-fluorobiphenyl)-benzo[1,2-b:4,5-b′]-bithiophene (F2-BPTBT), 2,5-di(4-4′-fluorobiphenyl)-thieno[3,2-b]thiophene (F2-BPTT), 5,5′-di(4-4′-fluorobiphenyl)-2,2′-bithiophene (F2-BP2T), 5,5″-di(4-4′-fluorobiphenyl)-2,2′:5′,2″-terthiophene (F2-BP3T), 5,5′″-di(4-4′-fluorobiphenyl)-2,2′:5′,2″:5″,2′″-tetrathiophene (F2-BP4T), 4,4′″-di(4-fluorophenyl-1,1′:4′,1″:4″,1′″:4′″,1″″:4″″,1″″-sexiphenyl (F2-p8P), 2,5-di(4-4″-fluoro-1,1′:4′,1″-terphenyl)-thiophene (F2-3PT), 5,5′-di(4-4″-fluoro-1,1′:4′,1″-terphenyl)-2,2′-bithiophene (F2-3P2T), 2,5-di(4-4″-fluoro-1,1′:4′,1″-terphenyl))-thieno[3,2-b]thiophene (F2-3PTT), 2,7-di(4-3′,5′-bifluorobiphenyl)-phenanthrene (F4-BPPh), 2,7-di(4-3′,5′-bifluorobiphenyl)-dibenzothiophen (F4-BPBTB), 2,6-di(4-3′,5′-bifluorobiphenyl)-benzo[1,2-b:4,5-b′]bithiophene (F4-BPTBT), 2,5-di(4-3′,5′-bifluorobiphenyl)-thieno[3,2-b]thiophene (F4-BPTT), 5,5′-di(4-3′,5′-bifluorobiphenyl)-2,2′-bithiophene (F4-BP2T), 5,5″-di(4-3′,5′-bifluorobiphenyl)-2,2′:5′,2″-terthiophene (F4-BP3T), 5,5′″-di(4-3′,5′-bifluorobiphenyl)-2,2′:5′,2″:5″,2′″-tetrathiophene (F4-BP4T), 4,4′″″-di(3,5-bifluorobiphenyl)-1,1′:4′,1″:4″,1′″:4′″,1″″:4″″,1′″″-sexiphenyl (F4-p8P), 2,5-di(4-3″,5″-bifluoro-1,1′:4′,1″-terphenyl)-thiophene (F4-3PT), 5,5′-di(4-3″,5″-bifluoro-1,1′:4′,1″-terphenyl)-2,2′-bithiophene (F4-3P2T), 2,5-di(4-3″,5″-bifluoro-1,1′:4′,1″-terphenyl)-thieno[3,2-b]thiophene (F4-3PTT), 5,5′″-diphenyl-2,2′:5′,2″:5″,2′″-tetrathiophene (P4T), 5,5′″-di(4-fluorophenyl)-2,2′:5′,2″:5″,2′″-tetrathiophene (F2-P4T), and 5,5′″-di(3,5-bifluorophenyl)-2,2′:5′,2″:5″,2′″-tetrathiophene (F4-P4T).

TABLE 5

| Inducing layer | semiconductor | |
|---|---|---|
| molecule | molecule | epitaxial relationship |
| BPPh | VOPc | incommensurate epitaxy |
| | | commensurate epitaxy |
| | TiOPc | incommensurate epitaxy |
| | | commensurate epitaxy |
| BPBTB | VOPc | incommensurate epitaxy |
| | | commensurate epitaxy |
| | PbPc | commensurate epitaxy |
| | | incommensurate epitaxy |
| BPTBT | VOPc | commensurate epitaxy |
| | | incommensurate epitaxy |
| | TiOPc | commensurate epitaxy |
| | | incommensurate epitaxy |
| BPTT | VOPc | commensurate epitaxy |
| | | incommensurate epitaxy |
| | TiOPc | commensurate epitaxy |
| | | incommensurate epitaxy |
| | PbPc | commensurate epitaxy |
| | | incommensurate |
| | | epitaxy |
| | SnPc | incommensurate |
| | | epitaxy |
| BP3T | VOPc | commensurate epitaxy |
| | | incommensurate epitaxy |
| | TiOPc | commensurate epitaxy |
| | | incommensurate epitaxy |
| | PbPc | commensurate epitaxy |
| | | incommensurate epitaxy |
| BP4T | VOPc | commensurate epitaxy |
| | | incommensurate epitaxy |
| | TiOPc | commensurate epitaxy |
| | | incommensurate epitaxy |
| P8P | VOPc | commensurate epitaxy |
| | | incommensurate epitaxy |
| | TiOPc | commensurate epitaxy |
| | | incommensurate epitaxy |
| 3PT | VOPc | commensurate epitaxy |
| | | incommensurate epitaxy |
| | TiOPc | commensurate epitaxy |
| | | incommensurate epitaxy |
| 3P2T | TiOPc | commensurate epitaxy |
| | | incommensurate epitaxy |
| | VOPc | commensurate epitaxy |
| | | incommensurate epitaxy |
| | PbPc | commensurate epitaxy |
| | | incommensurate epitaxy |
| 3PTT | VOPc | commensurate epitaxy |
| | | incommensurate epitaxy |
| | TiOPc | commensurate epitaxy |
| | | incommensurate epitaxy |
| F2-BPPh | VOPc | commensurate epitaxy |
| | | incommensurate epitaxy |
| | PbPc | commensurate epitaxy |
| | | incommensurate epitaxy |
| F2-BPBTB | VOPc | commensurate epitaxy |
| | | incommensurate epitaxy |
| | TiOPc | commensurate epitaxy |
| | | incommensurate epitaxy |
| F2-BPTT | TiOPc | commensurate epitaxy |
| | | incommensurate epitaxy |
| | VOPc | commensurate epitaxy |
| | | incommensurate epitaxy |
| | PbPc | commensurate epitaxy |
| | | incommensurate epitaxy |
| F2-BP2T | VOPc | incommensurate epitaxy |
| | | commensurate epitaxy |
| | TiOPc | commensurate epitaxy |
| | | incommensurate epitaxy |
| | PbPc | commensurate epitaxy |
| | | incommensurate epitaxy |
| | SnPc | commensurate epitaxy |
| | | incommensurate epitaxy |
| F2-BP3T | TiOPc | commensurate epitaxy |
| | | incommensurate epitaxy |
| | VOPc | incommensurate epitaxy |
| | | commensurate epitaxy |
| F2-BP4T | PbPc | incommensurate epitaxy |
| | | commensurate epitaxy |
| | VOPc | incommensurate epitaxy |
| | | commensurate epitaxy |
| | TiOPc | incommensurate epitaxy |
| | | commensurate epitaxy |
| F2-p8P | VOPc | incommensurate epitaxy |
| | | commensurate epitaxy |
| | TiOPc | commensurate epitaxy |
| | | incommensurate epitaxy |
| F2-3PT | VOPc | commensurate epitaxy |
| | | incommensurate epitaxy |
| | TiOPc | commensurate epitaxy |
| | | incommensurate epitaxy |
| F2-3P2T | VOPc | commensurate epitaxy |
| | | incommensurate epitaxy |
| | PbPc | commensurate epitaxy |
| | | incommensurate epitaxy |
| F2-3PTT | VOPc | commensurate epitaxy |
| | | incommensurate epitaxy |
| | SnPc | commensurate epitaxy |
| | | incommensurate epitaxy |
| F4-BPPh | VOPc | commensurate epitaxy |
| | | incommensurate epitaxy |
| | TiOPc | commensurate epitaxy |
| | | incommensurate epitaxy |
| F4-BPBTB | VOPc | commensurate epitaxy |
| | | incommensurate epitaxy |
| | TiOPc | commensurate epitaxy |
| | | incommensurate epitaxy |
| F4-BPTBT | VOPc | commensurate epitaxy |
| | | incommensurate epitaxy |
| | PbPc | commensurate epitaxy |
| | | incommensurate epitaxy |
| F4-BPTT | VOPc | incommensurate epitaxy |
| | | commensurate epitaxy |
| | TiOPc | incommensurate epitaxy |
| | | commensurate epitaxy |
| F4-BP2T | VOPc | commensurate epitaxy |
| | | incommensurate epitaxy |
| | PbPc | commensurate epitaxy |
| | | incommensurate epitaxy |
| F4-BP3T | VOPc | commensurate epitaxy |
| | | incommensurate epitaxy |
| | TiOPc | commensurate epitaxy |
| | | incommensurate epitaxy |
| F4-BP4T | VOPc | commensurate epitaxy |
| | | incommensurate epitaxy |
| | TiOPc | commensurate epitaxy |
| | | incommensurate epitaxy |
| F4-p8P | VOPc | commensurate epitaxy |
| | | incommensurate epitaxy |
| | TiOPc | commensurate epitaxy |
| | | incommensurate epitaxy |
| F4-3PT | VOPc | commensurate epitaxy |
| | | incommensurate epitaxy |
| | TiOPc | commensurate epitaxy |
| | | incommensurate epitaxy |
| F4-3P2T | VOPc | commensurate epitaxy |
| | | incommensurate epitaxy |
| | TiOPc | commensurate epitaxy |
| | | incommensurate epitaxy |
| F4-3PTT | VOPc | commensurate epitaxy |
| | | incommensurate epitaxy |
| | TiOPc | commensurate epitaxy |
| | | incommensurate epitaxy |
| P4T | TiOPc | commensurate epitaxy |
| | | incommensurate epitaxy |
| | PbPc | commensurate epitaxy |
| | | incommensurate epitaxy |
| | VOPc | commensurate epitaxy |
| | | incommensurate epitaxy |
| F2-P4T | VOPc | commensurate epitaxy |
| | | incommensurate epitaxy |
| | TiOPc | commensurate epitaxy |
| | | incommensurate epitaxy |
| | PbPc | commensurate epitaxy |
| | | incommensurate epitaxy |
| F4-P4T | TiOPc | commensurate epitaxy |
| | | incommensurate epitaxy |
| | VOPc | commensurate epitaxy |
| | | incommensurate epitaxy |

The present invention is not limited to the above-mentioned embodiments. In general, the inducing layer materials for the use of Weak Epitaxy Growth disclosed in the present invention can be used in other organic semiconductor components to prepare components in two-dimensional or three-dimensional integrated device. These integrated device can be used in integrated circuits, active matrix display, sensors and photovoltaic cells. The electronic components based on the present invention are easy to be manufactured in large scale.

1. Inducing layer materials for the weak epitaxial films of non-planar metal phthalocyanine represented by the following general formula [I]: 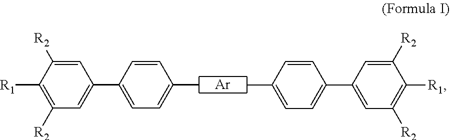
Wherein Ar is a conjugated aromatic group, including the following structures: 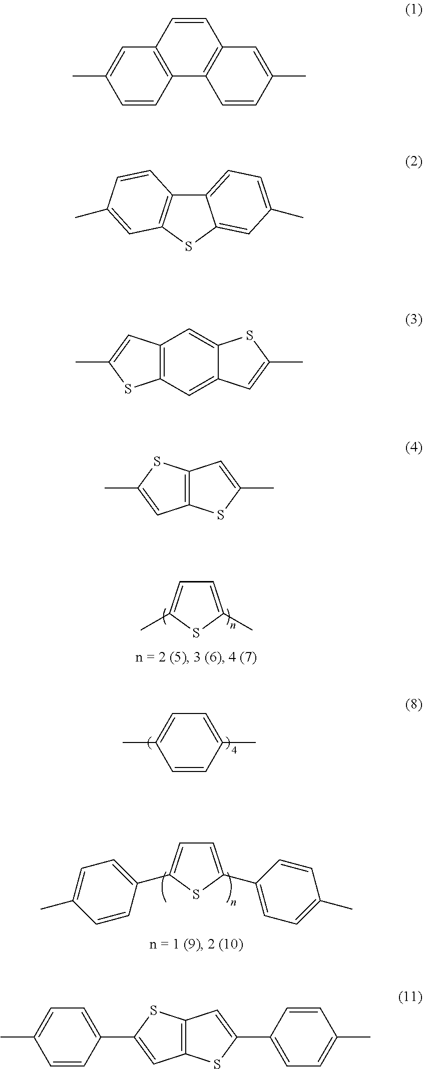
R₁ and R₂ in general formula I is hydrogen atom (H) or fluorine atom (F), according to the differences of the chemical structures of R₁ and R₂, the molecular structures of the new inducing layer materials conformed to general formula I can be divided into the following three types: (1) R₁═R₂═H, and a case where Ar represents the structure of (5) is excluded, (2) R₁═F, R₂═H, (3) R₁═H, R₂═F.

2. Inducing layer materials according to claim 1, characterized in that, the structures of the said inducing layer materials are as follows:
any one of 2,7-di(4-biphenyl)-phenanthrene (BPPh), 2,7-di(4-biphenyl)-dibenzothiophen (BPBTB), 2,6-di(4-biphenyl)-benzo[1,2-b:4,5-b ′]bithiophene (BPTBT), 2,5-di(4-biphenyl)thieno[3,2-b]thiophene (BPTT), 5,5″-di(4-biphenyl)-2,2′:5′,2″-terthiophene (BP3T), 5,5′″-di(4-biphenyl)-2,2′:5′,2″:5″,2′″-tetrathiophene (BP4T), 1,1′:4′,1″:4″,1′″:4′″,1″″:4″″,1′″″:4′″″,1″″″:4″″″,1′″″″-octiphenyl (p8P), 2,5-di(4-1,1′:4′,1″terphenyl)-thiophene (3PT), 5,5′-di(4-1,1′:4′,1″-terphenyl)-2,2′-bithiophene (3P2T), 2,5-di(4-1,1′:4′,1″-terphenyl)-thieno[3,2-b]thiophene (3PTT), 2,7-di(4-4′-fluorobiphenyl)-phenanthrene (F2-BPPh), 2,7-di(4-4′-fluorobiphenyl)-dibenzothiophen (F2-BPBTB), 2,6-di(4-4′-fluorobiphenyl)-benzo[1,2-b:4,5-b′]bithiophene (F2-BPTBT), 2,5-di(4-4′-fluorobiphenyl)-thieno[3,2-b]thiophene (F2-BPTT), 5,5′-di(4-4′-fluorobiphenyl)-2,2′-bithiophene (F2-BP2T), 5,5″-di(4-4′-fluorobiphenyl)-2,2′:5′,2″-terthiophene (F2-BP3T), 5,5′″-di(4-4′-fluorobiphenyl)-tetrathiophene (F2-BP4T), 4,4′″″-di(4-fluorophenyl-1,1′:4′,1″:4″,1′″:4′″,1″″:4″″,1′″″-sexiphenyl (F2-p8P), 2,5-di(4-4″-fluoro-1,1′:4′,1″-terphenyl)-thiophene (F2-3PT), 5,5′-di(4-4″-fluoro-1,1′:4′,1″-terphenyl)-2,2′-bithiophene (F2-3P2T), 2,5-di(4-4″-fluoro-1,1′:4′,1″-terphenyl))-thieno[3,2-b]thiophene (F2-3PTT), 2,7-di(4-3′,5′-bifluorobiphenyl)-phenanthrene (F4-BPPh), 2,7-di(4-3′,5′-bifluorobiphenyl)-dibenzothiophen (F4-BPBTB), 2,6-di(4-3′,5′-bifluorobiphenyl)-benzo[1,2-b:4,5-b′]bithiophene (F4-BPTBT), 2,5-di(4-3′,5′-bifluorobiphenyl)-thieno[3,2-b]thiophene (F4-BPTT), 5,5′-di(4-3′,5′-bifluorobiphenyl)-2,2′-bithiophene (F4-BP2T), 5,5″-di(4-3′,5′-bifluorobiphenyl)-2,2′:5′,2″-terthiophene (F4-BP3T), 5,5′″-di(4-3′,5′-bifluorobiphenyl)-2,2′:5′,2″:5″,2′″-tetrathiophene (F4-BP4T), 4,4′″″-di(3,5-bifluorobiphenyl)-1,1′:4′,1″:4″,1′″:4′″,1″″:4″″,1′″″-sexiphenyl (F4-p8P), 2,5-di(4-3″,5″-bifluoro-1,1′:4′,1″-terphenyl)-thiophene (F4-3PT), 5,5′-di(4-3″,5″-bifluoro-1,1′:4′,1″-terphenyl)-2,2′-bithiophene (F4-3P2T), 2,5-di(4-3″,5″-bifluoro-1,1′:4′,1″-terphenyl)-thieno[3,2-b]thiophene (F4-3PTT).

3. Inducing layer materials for the weak epitaxial films of non-planar metal phthalocyanine, characterized in that, the structures of the said inducing layer materials are as follows:
5,5′″-diphenyl-2,2′:5′,2″:5″,2′″-tetrathiophene (P4T), 5,5′″-di(4-fluorophenyl)-2,2′:5′,2″:5″,2′″-tetrathiophene (F2-P4T), or 5,5′″-di(3,5-bifluorophenyl)-2,2′:5′,2″:5″,2′″-tetrathiophene (F4-P4T).
